(19) United States
(12) Patent Application Publication
(10) Pub. No.: US 2004/0137643 A1
(43) Pub. Date: Jul. 15, 2004

(54) Immunodetection and quantification methods

(76) Inventor: John Humphreys, Irving, TX (US)

Correspondence Address: BARNES & THORNBURG, 11 SOUTH MERIDIAN, INDIANAPOLIS, IN, 46204

(21) Appl. No.: 10/725,891

(22) Filed: Dec. 2, 2003

Related U.S. Application Data
(60) Provisional application No. 60/430,189, filed on Dec. 2, 2002.

(51) Int. Cl. G01N 33/543

(52) U.S. Cl. 436/518

(57) ABSTRACT

Methods and kits are provided for the detection and quantification of a protein. The methods comprise immobilizing the protein of interest on a substrate, the protein comprising an epitope and another portion, contacting the immobilized protein with a labeled ligand that binds to the epitope to yield a detectably labeled protein, detecting the detectably labeled protein, and quantifying the detectably labeled protein, and the kits comprise a sample of a standard protein, wherein the standard protein comprises an epitope and the sample is of a known concentration of the standard protein, and an aliquot of a labeled primary antibody having specificity for the epitope.

This application claims the benefit of U.S. Provisional Application No. 60/430,189, filed Dec. 2, 2002, which is herein incorporated by reference.

### BACKGROUND AND SUMMARY OF THE INVENTION

Western blots have provided researchers with a sensitive technique to determine the presence of a specific protein, provided one has an appropriate antibody that specifically recognizes the target protein. However, obtaining an appropriate antibody with the appropriate level of specificity can be difficult. One solution is to “tag” recombinantly expressed proteins by incorporation of a specific amino acid sequence that serves as a recognition site for a generally available antibody, illustratively a commercially available antibody. This approach allows researchers to purchase an antibody that will facilitate the identification of any “tagged” recombinant protein by western blotting. Western blots have traditionally been considered an unacceptable technique for protein quantification. This is largely due to the complexity introduced by the multiple steps involved in conventional immunoblotting. In brief, proteins are first transferred into a membrane support, then are incubated with a primary antibody that recognizes the target protein. Unbound antibody is washed away, then the target protein and primary antibody are incubated with a secondary antibody that recognizes the primary antibody. The secondary antibody is then washed away, and often a tertiary antibody against the secondary antibody is incubated with the other proteins, and then unbound tertiary antibody is washed away. Finally, an enzyme (or other signal) attached to the last antibody is used to detect the presence of the antibody sandwich (and thus the original target protein), typically by the production of light or a dark blue dye.

Each additional step using another antibody or enzyme is used to amplify detection of the target protein associated with the primary antibody binding to the target protein. This process gives western blots exquisite sensitivity, but with each amplification step, error as well as signal is amplified. Antibodies typically come from natural sources and considerable variation from one rabbit, goat, or chicken to another is inherent. In addition, other variables including the age of the reagents used in the assay or differences in antibody dilution or buffer temperature can have dramatic effects on results from one blot to another. Because of this variability, western blotting is generally considered to be unreliable for protein quantification.

The illustrative Midwestern blot described herein eliminates or reduces many of the steps that can amplify errors, while still retaining the sensitivity of a traditional immunological blot. Thus, the Midwestern blot facilitates reliable and reproducible protein quantification. Midwestern blotting may also provide results in far less time than is possible using traditional western blotting, and the simplified procedure may make it much easier and faster for technicians or students to master the technique.

In an illustrative Midwestern blot, the target protein(s) are transferred into a membrane and the membrane is blocked as in a traditional western blot. An illustrative membrane is nitrocellulose or PVDF, although other membranes may be used, as are known in the art. The membrane and proteins are then incubated with a labeled primary ligand, illustratively with an inexpensive commercially available chromophore-labeled antibody. In an illustrative embodiment, anti-FLAG antibody labeled with a Cy3 chromophore is used to identify a FLAG-labeled P450. Unbound antibody is washed away and the membrane is allowed to dry. In an illustrative embodiment, no further manipulation performed on the blot, and no further hybridization with any other antibody is needed. The dry membrane can then be stored indefinitely or placed directly on the imaging surface of a variable-mode phosphor autoradiography imager such as the Typhoon instrument (Amersham Biosciences) and scanned. The Typhoon (or a similar instrument) was designed to detect storage phosphors exposed to radioactivity, but in the illustrative method, the Typhoon is used to detect the fluorescence of the chromophore-labeled antibodies bound to the target protein. Illustratively, the instrument's software is used to quantify signal from these antibodies, subtract background noise, and optionally provide the resulting data in a spreadsheet. In an illustrative method, protein quantification is accomplished by comparison of the signal from the target protein of interest to the signal of known amounts of other tagged proteins loaded on the same gel. The known standards act as an internal control reducing or eliminating many additional sources of inconsistency including those introduced during protein transfer or by variations in primary antibody concentration from one blot to another. While the Typhoon is used in an illustrative embodiment, detection devices such as or other imagers or densitometers may be used within the scope of this invention. Furthermore, it is understood that the choice of anti-FLAG antibody is illustrative, and other commercial and non-commercial antibodies may be used. Additionally, while the Cy3 chrompohore is used in the illustrative example, it is understood that other chromophores may be used. Alternatively, the antibody may be tagged with a wide variety of other labels, including but not limited to radioactive and enzymatic labels, as are known in the art. The choice of label need only be compatible with the detection method used.

Additional features of the present invention will become apparent to those skilled in the art upon consideration of the following detailed description of the preferred embodiments.

### BRIEF DESCRIPTION OF THE DRAWINGS

FIG. 1 is a schematic representation of FLAG-C4H and a P450 reductase, showing the N-terminal eight-amino acid FLAG epitope within the endoplasmic reticulum lumen, separated from the catalytic domain of the enzyme.

FIGS. 2A-C show detection of commercial FLAG-BAP and FLAG-C4H expressed in yeast. SDS-PAGE (FIG. 2A) and corresponding Midwestern blot (FIG. 2B) analysis of microsomal proteins from yeast harboring pYeDP60 void vector (Lane 1), pYeDP60-C4H (Lane 2) or pYeDP60-FC4H (Lane 3). Lane 4 contains commercial FLAG-BAP and BSA as carrier protein. FIG. 2C is a trace representing the average fluorescent signal for lanes 2, 3 and 4 of the Midwestern blot.

FIGS. 3A-B show analysis of the range of linear response for FLAG-BAP. FIG. 3A shows a Midwestern blot with increasing amounts FLAG-BAP in 2 mg mL⁻¹ BSA. FIG. 3B shows analysis of anti-FLAG antibody fluorescent signal from FLAG-BAP standards with (solid line) and without (dashed line) 2 mg mL⁻¹ BSA. (n=3±SD).

FIGS. 4A-B show analysis of the response for FLAG-C4H analyzed by Midwestern blotting. FIG. 4A shows a Midwestern blot with increasing amounts of microsomes containing FLAG-C4H. FIG. 4B is analysis of anti-FLAG antibody fluorescent signal from assays with increasing amounts of microsomes. (n=3±SD).

FIGS. 5A-B show quantification of FLAG-C4H. FIG. 5A is a Midwestern blot with three FLAG-BAP standards and FLAG-C4H in yeast microsomes, each run in triplicate. FIG. 5B is a three-point linear plot constructed from the FLAG-BAP signal, giving a standard response factor for FLAG-C4H quantification.

FIG. 6 shows carbon monoxide difference spectra of dithionite-reduced microsomes from yeast expressing native C4H, FLAG-C4H, or from yeast carrying the pYeDP60 void vector.

FIGS. 7A-B show Lineweaver-Burk kinetic analysis of native (FIG. 7A) and FLAG-C4H (FIG. 7B) catalyzed substrate para-hydroxylation of cinnamic acid. (n=3±SD).

### DETAILED DESCRIPTION OF THE INVENTION

Cytochrome P450-dependent monooxygenases (P450s) typically catalyze irreversible reactions in plant metabolite biosynthesis and animal catabolic metabolism. Consistent advances in our understanding of the roles played by P450s in key steps in steroid and xenobiotic (drug) metabolism in humans, as well as in plant secondary metabolism, have driven P450 research. P450s are typically expressed at low concentrations in vivo, and like many membrane-bound proteins, P450 yields from heterologous expression systems can be problematically low. This can be a significant obstacle to P450 reaserch.

Carbon monoxide (CO) difference spectroscopy has been used almost exclusively for P450 quantification for nearly four decades. This technique is based on a characteristic spectroscopic shift resulting from CO binding to the P450 heme prosthetic group (Peiscah et al., 1973; Loew and Rohmer, 1980). CO difference spectroscopy is rapid, and permits the direct measurement of catalytically active P450s without the need for enzyme purification, at least after expression in a heterologous host containing little or no endogenous P450s. Even though this technique has been proven to be robust and broadly applicable, an appreciable amount of P450 is needed for accurate measurements. Although the relatively high extinction coefficient of 91,000 cm⁻¹ M-1 of CO bound heme in P450s (Omura and Sato, 1964) facilitates spectroscopic quantification, this factor is frequently offset by the need to directly measure low P450 concentrations. Spectrophotometers used in molecular biology laboratories typically lack the accuracy and precision needed to quantify P450s from difference spectra of this magnitude. Higher concentrations of microsomes can be used to increase the amount of P450 in the lightpath, but this can result in increased light scatter from lipid vesicles. Various techniques used to enrich and solubilize P450s, such as Triton-X114 phase partitioning (Werck-Reichart et al., 1991), can be used to concentrate proteins to facilitate spectrophotometric detection, but these techniques also introduce additional sample manipulation, thus complicating P450 quantification.

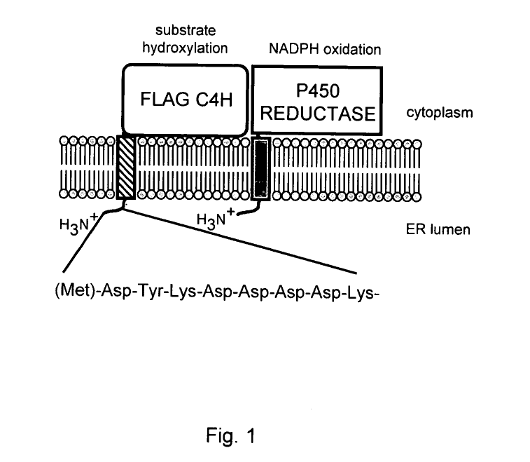

An alternative approach to P450 spectroscopy is the use of immunological techniques, which have the added benefit of being potentially applicable to any protein (Edwards et al., 1998; Stressor and Kupfer, 1999; Coorssen et al., 2002; Bergendahl et al., 2003). Such methods often involve secondary and possibly tertiary antibody binding, with inherent compounding of error. Moreover, if one wanted to compare many different P450s, the same antibody would not be expected to have similar affinities to multiple proteins. To address these issues, for heterologously expressed proteins, an epitope may be incorporated into the protein for which there is a corresponding commercially available monoclonal antibody; however, such an approach assumes that introduction of the epitope has no effect on the activity of the protein. This assumption is likely to be valid for most membrane-bound cytochromes P450, as these enzymes are thought to have a conserved topology (Graham-Lowrence and Peterson, 1996) in which the catalytically active cytoplasmic domain is separated from the N-terminus of the protein by a single hydrophobic membrane anchor. Therefore, an epitope added to the amino-terminal end of a P450, illustratively the eight-amino acid FLAG-tag epitope (Hopp et al., 1988), would be separated from the catalytic domain by the endoplasmic reticulum membrane and would, therefore, be unlikely to alter the overall enzymatic properties of the P450 or its interactions with other cytoplasmic proteins (FIG. 1). The P450 system thus provides an excellent model system to test detection and quantification via the illustrative Midwestern blot.

The availability of commercial chromophore-labled monoclonal antibodies, coupled with the development of bench-top instruments capable of fluoresence detection and quantification, have facilitated the development of the herein described quantification technique. The present methods using recombinant P450 quantification preserve many of the advantages of CO difference spectroscopy, including the ability to measure P450 content in crude microsomes, and to quantify many P450s with the same technique. In an illustrative embodiment, a single labeled primary antibody is used, requiring fewer steps and less time than conventional protein immunoblotting protocols often referred to as western blotting. Further, the illustrated method permits FLAG-tagged protein quantification by comparison of signal to that of a commercially available FLAG-tagged external standard. The modified and truncated western blot, developed in the Midwest, is herein referred to as the Midwestern or fluorescent-labeled antibody probe (FLAP) blot. Midwestern blots can have a significantly lower limit of quantification, illustrateively a 400 fold lower limit of quantification than traditional CO difference spectroscopy, allowing P450 quantification well below the limits of traditional CO difference spectroscopy.

While reference is made to P450 quantification, it is understood that the methods described herein may be used to quantify a wide variety of proteins. While not so limited, the methods described herein are well suited for heterologously expressed proteins having a domain or terminus that is separated from the active domain of the protein and whose activity is likely to be generally unaffected by the introduction of an epitope into that domain or epitope. Furthermore, while reference is made to the FLAG epitope, it is understood that any epitope may be used, with epitopes having commercially available antibodies being particularly convenient.

### EXPERIMENTAL PROCEDURES

Yeast P450 Expression Constructs

For the construction of the pYeDP60-C4H and pYeDP60-FC4H expression plasmids, the Arabidopsis C4H open reading frame was amplified by PCR using Takara Extaq (Panvera, Madison, Wis.) with one of two upstream primers, 5′-GAAGTCCAAGCTTGACCTCCTCTTGCTGGAGAAG-3′(SEQ ID NO:1) for the P450 cinnamate 4-hydroxylase (C4H) and 5′-CTTCTCGGATCCATGGACCTCCTCTTGCTGGAGAAG-3′ (SEQ ID NO:2) for amino-terminal FLAG-epitope tagged C4H (FLAG-C4H), in combination with the downstream primer 5′-TGAAACCAAGAATTCTTAACAGTTCCTTGGTTTCA-3′ (SEQ ID NO:3). The 5′ primers introduce a BamHI site one codon upstream, or a HindIII site one codon downstream, respectively, of the native start codon and the 3′ primer introduces an EcoRI site downstream of the stop codon. To eliminate possible Taq-introduced errors, the resulting PCR products were subcloned into pGEM T-Easy (Promega, Madison, Wis.) and a SacII-StyI cassette spanning most of the amplified region was removed and replaced with corresponding sequence from the original cDNA clone. These constructs were confirmed by sequencing. To generate pYeDP60-C4H, the subcloned C4H open reading frame was digested to yield BamHI-SacII and SacII-EcoRI fragments, which were introduced into BamHI-EcoRI digested pYeDP60 (Urban et al, 1990) in a three-way ligation. To generate pYeDP60-FC4H, two complementary oligonucleotides containing a methionine start codon followed by the appropriate codons for the eight-amino acid FLAG epitope were designed with 5′ BamHI and 3′ HindIII overhangs

| | |
|---|---|
| (5′-GATCCAAAATGGACTACAAGGATGACGAT | (SEQ ID NO: 4) |
| GACA-3′ and | |
| 5′-ACGTTGTCATCGTCATCCTTGTAGTCCAT | (SEQ ID NO: 5)) |
| TTTG-3′. | |

three-way ligation was used to ligate the FLAG linker and the HindIII-EcoRI C4H ORF into Bam HI-EcoRI digested pYeDP60 to yield the plasmid pYeDP60-FC4H. The resulting C4H expression vectors were confirmed by sequencing.

Yeast Strains and Transformation

The construction of the Saccharomyces cerevisiae strain WAT11, a derivative of the W303-B strain (MATa; ade2-1; his3-11,-15; leu2-3,-112; ura3-1; can^{R}; cyr⁺) expressing the ATR1 Arabidopsis NADPH-P450 reductase has been previously described Pompon et al., 1996; Urban et al., 1997). WAT11 cells were transformed with pYeDP60, pYeDP60-C4H and pYeDP60-FC4H essentially as described by Giets et al., (1992).

Yeast Growth Conditions and Preparation of Yeast Microsomal Extracts

WAT11 cells harboring pYeDP60, pYeDP60-C4H and pYeDP60-FC4H were grown in flasks shaken at 250 RPM at 30° C., then induced with 20 g L⁻¹ galactose and shaken at 150 RPM at 24° C. until reaching an OD of 2.8. Microsomes were then prepared essentially as described by Urban et al., (1994). Yeast cells were washed in lysis buffer (50 mM Tris-HCl, pH 7.4, containing 20% glycerol, 4 mM EDTA and 150 mM NaCl), then broken in lysis buffer by vortexing with 0.5 mm zirconia beads (Biospec Products, Bartlesville, Okla.). The preparation was centrifuged at 1000×g and the supernatant was further clarified by centrifugation at 10,000×g. One hundred mg of PEG 3350 was then added per mL of buffer and the mixture was incubated for 15 min with stirring at 4° C., followed by centrifugation at 10,000×g. The microsomal pellets were resuspended in assay buffer (50 mM Tris-HCl, pH 7.4, containing 20% glycerol, 4 mM EDTA) using a dounce homogenizer, then stored at −70° C.

Microsome and FLAG-BAP Quantification

Protein content in yeast microsomes was determined with the bicinchonic acid assay procedure (Pierce, Hercules, Calif.) with bovine serum albumin (BSA) (Sigma, St. Louis, Mo.) as a standard (Lowrey et al., 1951; Morton and Evans, 1992). Amino-terminal FLAG epitope-tagged BAP (FLAG-BAP)(Sigma, St. Louis, Mo.) (Brizzard et al., 1994) was quantified by the Biorad protein assay (BioRad, Hercules, Calif.) (Bradford, 1976) with immunoglobulin G as a standard.

SDS PAGE and Immunoblots

Microsomal proteins were separated on 10% (w/v) polyacrylamide gels and transferred overnight (20 mM Tris, 150 mM glycine and 20% methanol buffer at 4° C. with 200 mA, 40 V) to Westran two micron pore size PVDF membrane (Schleicher & Schuell, Keene, N.H.) for immunoblot analysis (Laemmli, 1970; Tobin et al., 1979). All incubation steps were conducted on a rocker table. Blocking was performed using three 30 min incubations in PBS buffer (10 mM KH₂PO₄, pH 7.4, 0.85% NaCl and 0.05% Tween 20) containing 3% (w/v) powdered milk. Membranes were then incubated for one hour with anti-FLAG M2 monoclonal antibody-Cy3 conjugate (Sigma, St. Louis, Mo.) at a dilution of 1:125 in blocking solution. Finally, each PVDF membrane was separately washed using three 30 min incubations in PBS buffer, then allowed to dry.

Immunological Detection of Flag-Tagged Protein

Detection of bound anti-FLAG antibody was conducted using a Typhoon 8600 variable mode imager (Molecular Dynamics, Sunnyvale, Calif.). The immunoblot membrane was placed directly on the platen of the imager, and scanned for fluorescence using the 523 nm laser emission and 580 nm filtered fluorescence detection with the photomultiplier tube voltage at 500 V and a medium sensitivity setting. The resulting data were analyzed using ImageQuant software (Molecular Dynamics, Sunnyvale, Calif.). Quantitative analysis of the data was accomplished by averaging the fluorescence response of each row of pixels the width of the SDS PAGE lane from the top to the bottom of the gel. This information was displayed as a trace with detector response versus pixel as the abscissa and ordinate, respectively. Bands of anti-FLAG antibody bound to protein were visualized as peaks, with the area of these peaks determined by integration using a running baseline.

Spectroscopy

The carbon monoxide difference spectrum of wild-type and FLAG-C4H was measured by diluting yeast microsomes 1:5 in assay buffer containing 0.5% Triton X-100 to minimize optical density and light scattering. Microsomes were reduced with sodium dithionite, 1 mL aliquots were divided into matching cuvettes, and a baseline difference spectrum from 350 to 550 nm was measured using a Shimadzu UV2100 (Kyoto, Japan) with a bandwidth of 1 nm. Carbon monoxide gas was slowly bubbled through the experimental cuvette and a CO difference spectrum was recorded (Estabrook and Werringloer, 1978).

Enzyme Assays

C4H assays were carried out in the presence of various concentrations of cinnamic acid as previously described (Humphreys et al., 1999). Briefly, an NADPH regenerating system consisting of 1 mM NADP⁺, 10 mM glucose-6-phosphate, and 1 unit of glucose-6-phosphate dehydrogenase (Sigma, St. Louis, Mo.) was pre-incubated at 30° C. for 5 min to generate NADPH in a final volume of 450 PL of assay buffer. Assays were initiated by the addition of 50 μL of appropriately diluted microsomes and were incubated at 30° C. for 20 min before the reactions were stopped with 100 μL of 3N HCl. Samples were centrifuged for 20 min at 14,000×g and then analyzed by HPLC (Shimadzu Corp., Kyoto, Japan) using a Microsorb-MV C-18 column (Varian Analytical Instruments, Walnut Creek, Calif.) using the following method: solvent A, 1.5% acetic acid in water; solvent B, acetonitrile; flow rate of 1 mL min⁻¹; 0 to 11% B in 1.5 min, 1.5 to 35% B in 11.5 min, 35 to 100% B in 1 min, 100% B for 5 min. Reaction product was quantified by UV detection at 308 nm. Assays conducted with microsomes isolated from yeast transformed with pYeDP60 served as negative controls.

### RESULTS

Immunoblot Detection of Flag-C4H by Midwestern Blot

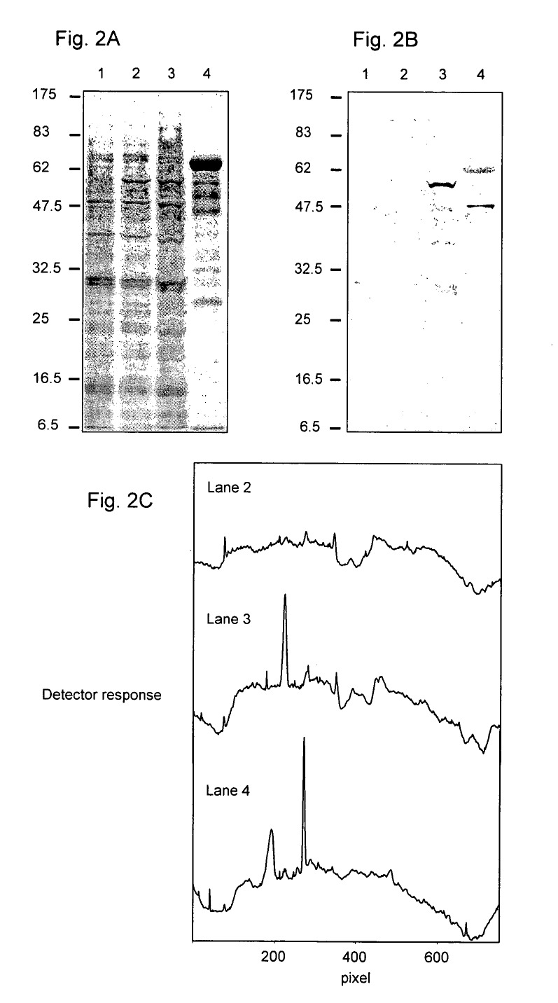

The detection of FLAG-tagged proteins using traditional techniques employing anti-FLAG antibodies and enzyme linked secondary antibodies have been described previously (Prickett et al., 1989; Ro et al., 2001). In contrast, the use of a Cy3-conjugated anti-FLAG monoclonal antibody for direct detection and quantification of FLAG-tagged proteins represents a significant departure from standard methods. To evaluate the utility of epitope tagging for the quantification of P450s in complex protein mixtures, microsomes from yeast expressing native or FLAG-C4H, as well as control microsomes, were compared to FLAG-BAP standards. Yeast microsomal proteins were separated by SDS PAGE and either transferred overnight to PVDF membrane for subsequent immunological analysis, or visualized directly by Coomassie staining. Protein staining revealed an induced 58 kDa band corresponding to C4H that was present in microsomes from yeast expressing C4H and FLAG-C4H, but was absent in the control microsomes suggesting that addition of the N-terminal FLAG epitope did not effect the subcellular localization of the protein (FIG. 2A). The 45 kDa FLAG-BAP standard is obscured by the presence of added carrier proteins composed primarily of BSA. Immunodetection of FLAG-tagged proteins by Midwestern blotting detected two prominent FLAG-tagged proteins: a 58 kDa protein that runs with the observed induced FLAG-C4H, and a 45 kDa band that runs with FLAG-BAP (FIG. 2B). No prominent signals were found when analyzing native C4H or control microsomes, although there was some weak, non-specific binding of the Cy3-conjugated antibody to abundant proteins in the yeast microsomes and the BSA carrier protein. There was no evidence that either FLAG-BAP or FLAG-C4H passed through the PVDF membrane onto a second or third stacked PVDF membrane (data not shown). Measuring the relative signal intensity and the range of linear response for two different proteins, FLAG-BAP and FLAG-C4H, provided an opportunity to evaluate many immunoblotting variables. Both nitrocellulose and PVDF membranes were found to be acceptable, but PVDF was preferred due to slightly lower autofluorescence. With these two proteins, it was found that variations in protein transfer technique, including methanol concentration in the transfer buffer, voltage, and extended transfer time appeared to have a minimal effect on the final signal intensity. The use of smaller well sizes for protein electrophoresis appeared to lower the limits of protein detection. In contrast, increasing sample size was not preferred because distortion resulting from protein and/or lipid overloading broadens the resulting protein band, making accurate integration more difficult.

ImageQuant software data analysis provides several ways to measure the fluorescent signal from Cy3-labeled antibodies. Cy3 signal can be measured by counting the total fluorescent signal in defined areas encompassing regions of interest on the membrane, then subtracting the background signal from an equivalent “control” area of the membrane. This approach can be problematic because the choice of the “control” area of the membrane adds significantly to result variability (data not shown). In contrast, visualizing the average of the fluorescent intensity in each lane as a whole produced good results. The output of this method is a trace with the abscissa representing the distance from the top of the membrane and the ordinate representing average fluorescence signal. Peaks in this trace represent fluorescent bands that can be integrated with a running baseline that acts as a local measure of background signal (FIG. 2C). This approach facilitates identification of signals that might otherwise be difficult to recognize by visual inspection.

Quantification of FLAG-C4H by Midwestern Blot

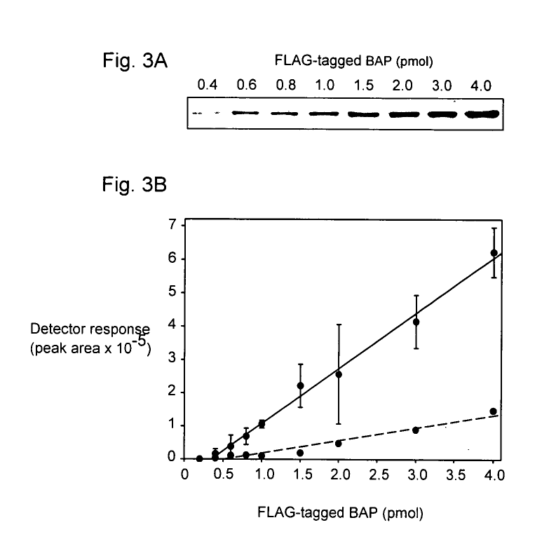

To determine the limits of quantification and the range of linear response for the detection of FLAG-tagged proteins by Midwestern blotting, increasing amounts of FLAG-BAP were analyzed on multiple blots run simultaneously. Initial experiments using FLAG-BAP standards showed a loss of signal which was most pronounced at low concentrations of FLAG-BAP, probably due to nonspecific binding of the protein to the plasticware used in sample preparation. The inclusion of BSA minimized this effect, and led to a linear response from 0.4 to over 4.0 pmol FLAG-BAP (FIGS. 3A-B).

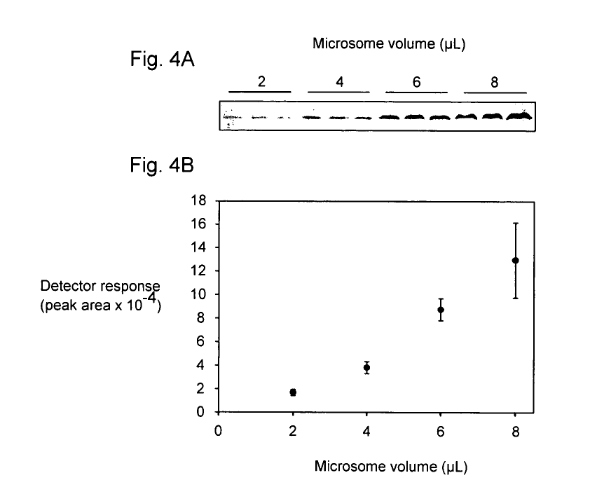

Increasing amounts of microsomes from yeast expressing FLAG-C4H were analyzed to determine the limits of detection and the range of linear response for FLAG-C4H (FIGS. 4A-B). A linear response was observed when between 4 and 8 μL of microsomes from yeast expressing FLAG-C4H were analyzed, with a slight deviation from linearity observed when lower levels of protein was tested. The level of FLAG-C4H in 2 μL of microsomes was detectable, but approached a signal to noise ratio of 10:1, near the limits of reliable quantification.

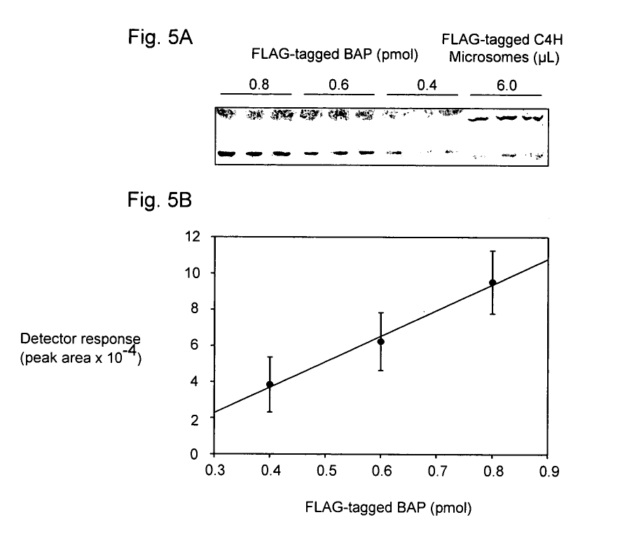

Having established the responses for proteins to be analyzed with this system, two approaches were taken to quantify FLAG-C4H in relation to a known amount of FLAG-BAP. Initially, known amounts of FLAG-BAP were added as an internal standard to FLAG-C4H-containing yeast microsomes. This approach has the advantage of placing the internal control as close as possible to the protein being measured. Unfortunately, a band in the yeast extracts running just below FLAG-BAP (FIGS. 2B and 2C) that is non-specifically recognized by the anti-FLAG antibody made signal integration problematic. To avoid this problem, a second approach was used in which FLAG-BAP standards and microsomes with an unknown amount of FLAG-C4H were assayed in separate lanes in triplicate (FIG. 5A). A simple three-point linear plot was constructed from the FLAG-BAP signal, giving a standard response factor. The signal from 6 μL aliquots of microsomes containing FLAG-C4H was measured in triplicate and quantified in comparison to this response factor (FIG. 5B). Using this method, 0.10±0.009 pmol μL⁻¹ FLAG-C4H was detected in microsomes of yeast carrying pYeDP60-FC4H (Table 1).

TABLE 1
Quantification and kinetic analysis of C4H and FLAG-tagged C4H.
SD, standard deviation.

| | P450 quantification | | | | |
|---|---|---|---|---|---|
| | (pmol μL⁻¹ microsomes ± SD) | | | Turnover (min⁻¹ ± SD) | |
| | CO difference | Midwestern | Kₘ ^{app.} | CO difference | Midwestern |
| Sample | spectrum | blot | (±SD) | spectrum | blot |
| Wild type C4H | 0.47 ± 0.04 | NA | 0.7 ± 0.01 | 28 ± 0.1 | NA |
| FLAG-tagged | 0.43 ± 0.04 | 0.10 ± 0.009 | 1.1 ± 0.09 | 83 ± 2 | 380 ± 7 |
| C4H | | | | | |

Quantification of C4H and Flag-C4H by CO Difference Spectroscopy

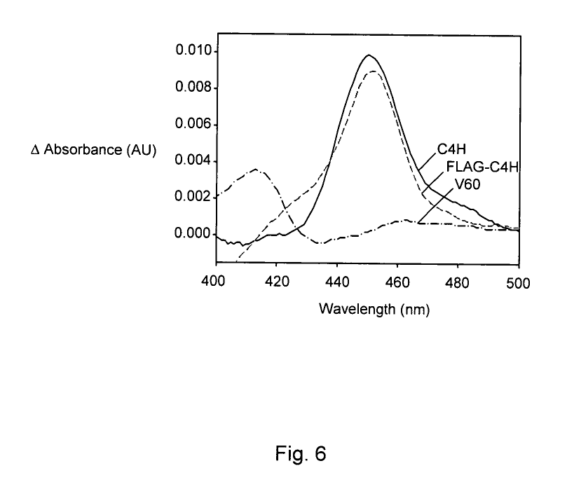

To quantify protein levels using conventional methods and to compare the folding and stability of native and FLAG-C4H, reduced carbon monoxide difference absorption spectra of both P450s were evaluated. Yeast microsomes containing either C4H or FLAG-C4H displayed a characteristic CO difference absorption peak at 450 nm (FIG. 6). No difference absorption peak at 420 nm was detected. The concentration of spectrally detectable native and FLAG-C4H were found to be approximately 0.47±0.04 and 0.43±0.04 pmol μL⁻¹ microsomes, respectively (Table 1), suggesting that the FLAG epitope in the FLAG-C4H protein did not have a major impact on P450 production, folding or targeting. The signal to noise ratio for these measurements was approximately 5:1, a reliable limit of quantification for CO difference spectroscopy.

Catalytic Properties of C4H and FLAG-C4H

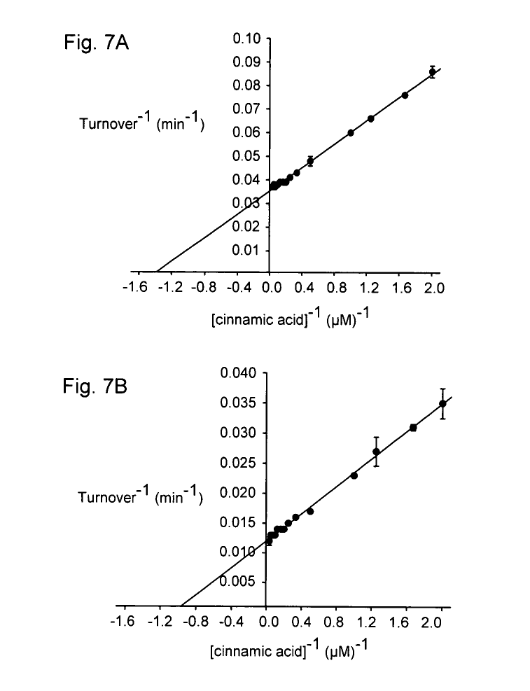

To evaluate the impact of introducing an amino-terminal FLAG epitotope on C4H enzymatic activity, kinetic parameters of both native and FLAG-C4H were determined using the same microsomal fractions used for protein quantification. It is important to note that the WAT11 yeast strain was chosen because this strain is engineered to express the Arabidopsis P450 reductase gene ATR1 under the control of the same galactose promoter used to drive P450 expression, eliminating the need to reconstitute the P450/P450 reductase system. Consistent with subcellular targeting of wild-type C4H expressed in yeast, both the native and FLAG-C4H activity was found in the microsomal fraction. C4H and FLAG-C4H dependent p-coumarate production was found to follow characteristic Michaelis-Menten behavior (FIGS. 7A-B). No p-coumarate formation was observed with microsomes from control yeast. Native and FLAG-C4H were found to have a Kₘ^{aPP} of 0.7±0.01 and 1.1±0.09 μM, respectively (Table 1), which is consistent with C4H Kₘ^{aPP} reports in the literature that vary from 1.8 to 8.2 μM (Urban et al., 1994; Mizutanti et al., 1997; Pierrel et al., 1994; Urban et al., 1997). Turnover rates of native and FLAG-C4H were also evaluated, using both spectroscopic and immunological P450 quantitation. When calculated using P450 concentrations obtained from CO difference spectroscopy, native C4H and FLAG-C4H turnover rates were determined to be 28±0.1 and 83±2.0 min¹, respectively. This difference, although significant, may be associated with variability in reductase activity in this heterologous expression system (data not shown). In comparison to CO difference spectroscopy, the lower concentration of FLAG-C4H obtained by Midwestern blot gives a correspondingly higher turnover rate of 380±7 min⁻¹. Taken as a whole, kinetic analysis of native and FLAG-C4H suggests that the addition of an amino-terminal FLAG epitope has little effect on C4H catalytic activity.

### DISCUSSION

Evaluation of P450 activity, expression and purification can necessitate quantification of small amounts of protein. The Midwestern blot, a simplified variation of a western blot, is herein described which is useful for identifying and quantifying heterologously expressed proteins that include an appropriate introduced epitope. P450s are good model proteins for evaluating Midwestern blotting because they can be quantified by both spectroscopic and immunogenic techniques. Midwestern blots require fewer steps and thus less time, and produce readily quantifiable results making data interpretation easier than with a traditional western blot, and can be archived. Moreover, commercially available labeled antibodies may be used, thus obviating the need for antibody derivatization and standardization. Further, since the antibodies employed in the illustrative embodiment are directed toward the well-characterized FLAG epitope, this epitope can be engineered into the recombinant protein of interest so that it can be used for direct quantitation by comparison to commercially available FLAG labeled protein standards.

The observed FLAG-C4H turnover rates of 83±2 and 380±7 min¹, calculated using spectroscopic or immunological quantification, respectively, are consistent with the variability seen in the published turnover rates ranging from 68 min⁻¹ (Mizutani et al., 1997) to 274 min⁻¹ Ro et al., 2001) for heterologously expressed C4H coupled with its native reductase. It is tempting to speculate that much of the variability in these reported rates, including those published here, stems from technical issues associated with using CO difference spectroscopy to calculate C4H turnover. It is expected that the reduced limits of detection and quantification of Midwestern blotting will facilitate research requiring the measurement of low amounts of P450 that would be impractical with CO difference spectroscopy.

In addition to comparing this technique with CO difference spectroscopy for P450 quantification, the effect of modifying the amino-terminal sequence of C4H by the addition of a FLAG epitope by measuring the kinetic properties of native and modified C4H has been evaluated. The results indicate that P450s are good candidates for amino-terminal epitope tagging. Both Midwestern blot and CO difference spectroscopy showed native and FLAG-C4H expression levels consistent with those obtained with other P450s in this yeast system (10-120 pmol mg⁻¹ microsomal protein) (Urban, et al., 1994). The absence of detectable P420 suggests FLAG-C4H is correctly folded and stable. In addition, Midwestern blotting revealed no inducible protein signals of a lower molecular weight than 58 kDa also suggesting that FLAG-C4H is correctly expressed and stable in yeast. Finally, while not identical, the observed Kₘ^{aPP} and activity of FLAG-C4H is similar to native C4H (Table 1) as well as to kinetic parameters reported for native C4H in the literature. These results strongly suggest that the amino-terminal addition of the eight amino acids that make up the FLAG epitope has little or no effect on the function of C4H.

Optimization of sample storage and handling can affect results. For instance, in the P450 system described herein, in the absence of BSA, FLAG-BAP loss, possibly due to association with plastic, had a dramatic effect on the linear response for FLAG-BAP, with the effect being more pronounced in samples with lower FLAG-BAP concentrations. The addition of a carrier protein, illustratively BSA, minimizes this effect, with FLAG BAP having a useful linear response at least from 0.4 to over 4.0 pmol. Such problems with signal linearity were not observed with FLAG-C4H, perhaps because P450s and their associated phospholipids have a lower affinity for plastics, or because the crude yeast microsomal fractions contain sufficient protein to minimize loss of FLAG-C4H.

The extinction coefficient first determined for P450 CO difference spectroscopy was derived from the total P450 content of rabbit liver microsomes (Omura and Sato, 1964). Because of the difficulty in obtaining cytochrome P450 extinction coefficients, the value obtained by Omura and Sato from rabbit livers is almost universally used with nearly all P450s. Considering the number of known prokaryotic, plant, and animal P450s that catalyze a diverse set of reactions on an untold number of substrates, it would not be surprising to find some variation in the extinction coefficients from one P450 to another. Midwestern blotting may provide a standard approach for the comparison of all heterologously expressed P450s.

Quantification of epitope-tagged P450s by Midwestern blot is advantageous for several reasons. First, although both Midwestern blotting and CO difference spectroscopy can quantify FLAG-C4H in crude extracts containing complex protein mixtures, Midwestern blotting requires significantly less P450 than CO difference spectroscopy. To quantify FLAG-C4H with a minimum of a 10:1 signal to noise ratio, 0.8 pmoles of FLAG-C4H are useful per assay with Midwestern blotting, whereas about 320 pmoles of FLAG-C4H are needed per assay to make the equivalent measurement using CO difference spectroscopy, for a 400-fold reduction in needed P450.

Second, Midwestern blotting allows P450 quantification where CO difference spectroscopy is not applicable. For example, allene oxide synthase is a P450 that has an a typically low affinity for CO, making quantitation by CO difference spectroscopy problematic (Lau et al., 1993). In contrast, Midwestern blotting is unaffected by P450 spectroscopic characteristics, or the type of expression system used to generate the enzyme, and is thus likely to be universally applicable to membrane-bound P450s. In addition, the quantification of individual P450s in organisms with substantial levels of endogenous P450s is impossible with CO difference spectroscopy, which measures total P450 concentration. In contrast, immunoblotting of epitope-tagged P450s can measure the concentrations of these enzymes in a complex background of other P450s, and could even be extended to detect multiple P450s, each carrying a unique epitope.

Third, the instruments and expertise required for Midwestern blotting may be available where spectroscopic expertise is not. As “genomics” transitions into “proteomics,” P450 research is now commonplace in laboratories that have considerably more experience with standard molecular biology tools, including western blots, than with spectroscopy. The Midwestern blot can provide investigators with a non-spectroscopic means of recombinant P450 quantification and an additional tool for P450 research.

Finally, it is understood that Midwestern blotting can be useful for the detection and quantification of any protein. The conserved topology of P450s, and the need to quantify these enzymes in crude extracts, make P450s an ideal application for Midwestern blotting; however, this technique should be applicable to any protein, illustratively proteins where the addition of an epitope tag can be shown not to have significant undesirable effects. Moreover, due to the ease and speed, Midwestern blots can be used for high throughput detection, whereby numerous samples may be run simultaneously, and numerous blots can be scanned simultaneously. Illustratively, 12 small membranes could be scanned simultaneously, each with 9 unknowns and 3 standards per gel, although other configurations are within the scope of this invention. Midwestern blotting is an additional tool that will complement the growing number of precipitation, detection, purification and in situ immunological strategies available to researchers working with epitope-tagged proteins.

### REFERENCES

Bergendahl, V., Glaser, B. T., Burgress, R. R., 2003. A fast Western blot procedure improved for quantitative analysis by direct fluorescence labeling of primary antibodies. J. Immunol. Methods. 277, 117-125.

Bradford, M. M. 1976. A rapid and sensitive method for the quantification of microgram quantities of protein utilizing the principle of protein-dye binding. Anal. Biochem. 72, 248-276.

Brizzard, B. L., Chubet, R. G., Vizard, D. L. 1994. Immunoaffinity purification of FLAG epitope-tagged bacterial alkaline phosphatase using a novel monoclonal antibody and peptide elution. BioTechniques. 16, 730-734.

Coorssen, J. R., Blank, P. S., Albertorio, F., Bezrukov, L., Kolosova, I., Blacklund, P. S. Jr., Zimmerburg, J. 2002. Quantitative fempto- to attomole immunodetection of regulated secretory vesicle proteins critical to exocytosis. Anal. Biochem. 307, 54-62.

Edwards, R. J., Adams, D. A., Watts, P. S., Davies, D. S., Boobis, A. R. 1998. Development of a comprehensive panel of antibodies against the major xenobiotic metabolizing forms of cytochrome P450 in humans. Biochem. Pharmacol. 56, 377-387.

Estabrook, R. W., Werringloer, J. 1978. The measurement of difference spectra: Application to the cytochromes of microsomes. Methods Enzymol. 52, 212-220.

Gietz, R. D., St. Jean, A., Woods, R. A., Schiestl, R. H. 1992. Improved method for high-efficiency transformation of intact yeast-cells. Nucleic Acids Res. 20, 1425.

Graham-Lorence, S., Peterson, J. A. 1996. P450s: Structural similarities and functional differences. FASEB J. 10, 206-214.

Hopp, T. P., Prickett, K. S., Price, V. L., Libby, R. T., March, C. J., Cerretti, D. P., Urdal, D. L., Conlon, P. J. 1988. A short polypeptide marker sequence useful for recombinant protein identification and purification. Bio/Technology. 6, 1204-1210.

Humphreys, J. M., Hemm, M. R., Chapple, C. 1999. New routes for lignin biosynthesis defined by biochemical characterization of recombinant ferulate 5-hydroxylase, a multifunctional cytochrome P450-dependent monooxygenase. Proc. Natl. Acad. Sci. USA. 96, 10045-10050.

Laemmli, U. K. 1970. Cleavage of structural proteins during the assembly of the head of bacteriophage. Nature. 227, 680-685.

Lau, S. C., Harder, P. A., O'Keefe, D. P. 1993. Low carbon monoxide affinity allene oxide synthase is the predominant cytochrome P450 in many plant tissues. Biochemistry. 32, 1945-1950.

Loew, G. H.,. Rohmer, M. M. 1980. Electronic spectra of model oxy, carboxy P450, and carboxy heme complexes. J. Am. Chem. Soc. 102, 3655-3657.

Lowry, O. H., Rosebrough, N. J., Farr, A. L., Randall, R. J. 1951. Protein measurement with the Folin phenol reagent. J. Biol. Chem. 193, 265-275.

Mizutani, M. Ohta, D. Sato, R. 1997. Isolation of a cDNA and a genomic clone encoding cinnamate 4-hydroxylase from Arabidopsis and its expression manner in planta. Plant Physiol. 113, 755-763.

Morton, R. E., Evans, T. A. 1992. Modification of the bicinchonic acid protein assay to eliminate lipid interference in determining lipoprotein protein content. Anal. Biochem. 204, 332-334.

Omura, T., Sato, R. 1964. The carbon monoxide-binding pigment of liver microsomes. II. Solubilization, purification, and properties. J. Biol. Chem. 239, 2379-2385.

Peisach, J., Stern, J. O., Blumberg, W. E., 1973. Optical and magnetic probes of the structure of cytochrome P-450's. Drug Metab. Dispos. 1, 45-61.

Pierrel, M. A., Batard, Y., Kazmaier, M., Mignotte-Vieux, C., Durst, F., Werck-Reichhart, D. 1994. Catalytic properties of the plant cytochrome P450 CYP73 expressed in yeast. Substrate specificity of a cinnamate hydroxylase. Eur. J. Biochem. 224, 835-844.

Pompon, D., Louerat, B., Bronine, A., Urban, P. 1996. Yeast expression of animal and plant P450s in optimized redox environments. Methods Enzymol. 272, 51-64.

Prickett, K. S., Amberg, D. P., Hopp, T. P., 1989. A calcium-dependent antibody for identification and purification of recombinant proteins. BioTechniques. 7, 580-586.

Ro, D. K., Mah, N., Ellis, B. E., Douglas, C. J. 2001. Functional characterization and subcellular localization of poplar (Populus trichocarpa×Populus deltoides) cinnamate 4-hydroxylase. Plant Physiol. 126 317-329.

Stressor, D. M., Kupfer, D. 1999. Monospecific antipeptide antibody to cytochrome P-450 2B6. Drug Metab. Dispos. 27, 517-525.

Tobin, H., Staehelin, T., Gordon, J. 1979. Electrophoretic transfer of proteins from polyacrylamide gels to nitrocellulose sheets. Procedure and applications. Proc. Natl. Acad. Sci. USA. 76, 4350-4354.

Urban, P., Cullin, C., Pompon, D. 1990. Maximizing the expression of mammalian cytochrome P-450 monooxygenase activities in yeast cells. Biochimie. 72, 463-472.

Urban, P., Mignotte, C., Kazmaier, M., Delorme, F., Pompon, D. 1997. Cloning, yeast expression, and characterization of the coupling of two distantly related Arabidopsis thaliana NADPH-cytochrome P450 reductases with P450 CYP73A5. J. Biol. Chem. 272, 19176-19186.

Urban, P., Werck-Reichhart, D., Teutsch, H. G., Durst, F., Regnier, S., Kazmaier, M., Pompon, D. 1994. Characterization of recombinant plant cinnamate 4-hydroxylase produced in yeast—kinetic and spectral properties of the major plant P450 of the phenylpropanoid pathway. Eur. J. Biochem. 222 843-850.

Werck-Reichhart, D., Benveniste, I., Teutsch, H., Durst, F., Gabriac, B. 1991. Glycerol allows low-temperature phase-separation of membrane-proteins solubilized in Triton-X 114—Application to the purification of plant cytochrome-P450 and cytochrome b5. Anal. Biochem. 197, 125-131.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

All patents and other publications cited herein are expressly incorporated by reference.

1. A method for the detection and quantification of a protein of interest comprising:
immobilizing the protein of interest on a substrate, the protein comprising an epitope and another portion;
contacting the immobilized protein with a labeled ligand that binds to the epitope to yield a detectably labeled protein;
detecting the detectably labeled protein; and
quantifying the detectably labeled protein.

2. The method of claim 1 wherein the ligand is a monoclonal or polyclonal antibody.

3. The method of claim 2 wherein the contacting step is not repeated with a second antibody.

4. The method of claim 2 wherein the protein is a recombinant protein tagged with an amino acid sequence that serves as a recognition site for the labeled antibody.

5. The method of claim 2 wherein the antibody is a chromophore-labeled antibody.

6. The method of claim 1 wherein the detecting step comprises detecting with a phosphor autoradiography imager.

7. The method of claim 1 wherein the quantifying step employs an internal standard.

8. The method of claim 7 wherein the internal standard is a second protein comprising the epitope and a second portion, wherein the second portion is different from the portion of the protein of interest.

9. A method for the detection of a protein comprising:
immobilizing a protein comprising an epitope on a substrate;
contacting the immobilized protein with a chromophore-labeled antibody that binds to the epitope to yield a detectably labeled protein; and
detecting the detectably labeled protein with a phosphor autoradiography imager,
wherein the contacting step is not repeated with a second antibody prior to the detecting step.

10. The method of claim 9 wherein the protein is a recombinant protein tagged with an amino acid sequence that serves as a recognition site for the labeled antibody.

11. The method of claim 9 further comprising the step of
quantifying the detected detectably labeled protein.

12. The method of claim 9 further comprising simultaneously performing the immobilizing, contacting, and detecting steps on a plurality of proteins for high throughput detection.

13. A method for detecting a P450 protein comprising:
providing a recombinant P450 protein comprising an epitope;
contacting the P450 protein with a labeled ligand that binds to the epitope to yield a detectably labeled P450 protein; and
detecting the detectably labeled P450 protein.

14. The method of claim 13 further comprising the step of
quantifying the detected detectably labeled P450 protein.

15. The method of claim 13 wherein the epitope is a non-native sequence provided at the amino terminus of the P450 protein.

16. The method of claim 15 wherein the epitope comprises a FLAG sequence.

17. The method of claim 16 wherein the ligand comprises an anti-FLAG M2 monoclonal antibody.

18. The method of claim 13 wherein the labeled ligand comprises a chromophore-labeled antibody.

19. The method of claim 18 wherein the chromophore comprises a fluorescent chromophore.

20. The method of claim 19 wherein the detecting step comprises detecting fluorescence using a phosphor autoradiography imager.

21. The method of claim 13 wherein the recombinant P450 protein is provided in a crude extract.

22. A delectably labeled molecular complex comprising:
a P450 molecule comprising a FLAG epitope; and
a chromophore-labeled ligand bound to the FLAG epitope.

23. The detectably labeled molecular complex of claim 22 wherein the chromophore-labeled ligand comprises an antibody.

24. The detectably labeled molecular complex of claim 23 wherein the antibody comprises an anti-FLAG M2 monoclonal antibody.

25. A kit for the detection and quantification of a protein comprising
a sample of a standard protein, wherein the standard protein comprises an epitope and the sample is of a known concentration of the standard protein, and
an aliquot of a labeled primary antibody having specificity for the epitope.

26. The kit of claim 25 further comprising an instruction manual for quantifying an unknown amount of a second protein, the second protein comprising the epitope, precast SDS-page gels, precut membrane, and reagents.

27. The kit of claim 25 wherein the labeled primary antibody is labeled with a chromophore.

28. The kit of claim 25 further comprising a plurality of additional samples of the standard protein, each additional sample comprising a different known concentration of the standard protein.

29. The kit of claim 25 further comprising
a plurality of samples of different standard proteins, wherein each standard protein comprises a different epitope, and each sample is of a known concentration of its respective standard protein, and
a plurality of additional aliquots of different labeled primary antibodies, each different labeled primary antibody having specificity for its respective epitope on one of the standard proteins.


SEQUENCE LISTING (5 sequences)

SEQ ID NO: 1 - DNA, 34 bp, artificial sequence
  artificial PCR primer
gaagtccaagcttgacctcctcttgctggagaag

SEQ ID NO: 2 - DNA, 36 bp, artificial sequence
  Primer for PCR amplification for introduction of FLAG epitope.
cttctcggatccatggacctcctcttgctggagaag

SEQ ID NO: 3 - DNA, 35 bp, artificial sequence
  artificial PCR primer
tgaaaccaagaattcttaacagttccttggtttca

SEQ ID NO: 4 - DNA, 33 bp, artificial sequence
  artificial sequence for cloning amplified into restriction sites onto vector
gatccaaaatggactacaaggatgacgatgaca

SEQ ID NO: 5 - DNA, 33 bp, artificial sequence
  artificial sequence for cloning amplified into restriction sites onto vector
acgttgtcatcgtcatccttgtagtccattttg